(19) United States
(12) Patent Application Publication
(10) Pub. No.: US 2008/0200474 A1
(43) Pub. Date: Aug. 21, 2008

(54) NOVEL FLUNARIZINE SALT FORMS AND METHODS OF MAKING AND USING THE SAME

(75) Inventors: Matthew Peterson, Hopkinton, MA (US); Julius Remenar, Framingham, MA (US); Carlos Sanrame, Lexington, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 12/015,694

(22) Filed: Jan. 17, 2008

Related U.S. Application Data
(60) Provisional application No. 60/890,030, filed on Feb. 15, 2007.

(51) Int. Cl. A61K 31/495; C07D 295/00; A61P 43/00

(52) U.S. Cl. 514/252.12; 544/399

(57) ABSTRACT

Disclosed herein are novel salts of flunarizine with improved properties. Also described herein are novel pharmaceutical compositions comprising such flunarizine salts, methods of making, and related methods of treatment.

### FIELD OF THE INVENTION

The present invention relates to novel salts of flunarizine with improved properties. The invention also provides methods of using novel flunarizine salts in an oral dosage pharmaceutical composition and related methods of treatment with novel flunarizine salts.

### BACKGROUND OF THE INVENTION

Flunarizine is an organic compound with the chemical name 1-cinnamyl-4-(di-p-fluorobenzhydryl)piperazine. Flunarizine is a calcium channel blocker and is used therapeutically as a vasodilator. The free form of flunarizine and a dihydrochloride salt of flunarizine are known in the art.

Synthesis of flunarizine and its dihydrochloride salt is disclosed in U.S. Pat. No. 3,773,939. Flunarizine dihydrochloride (e.g., SIBELIUM®) has been approved in several countries for the prophylaxis of classic or common migraine.

Flunarizine dihydrochloride (e.g., SIBELIUM®) requires special packaging to prevent water uptake during storage of the bulk form. It would be advantageous to develop a flunarizine form that is less hygroscopic than flunarizine dihydrochloride and could, therefore, be stored in bulk and manufactured into dosage forms under less demanding conditions. The present novel salts of flunarizine may be valuable in the preparation of non-hygroscopic crystalline forms of flunarizine.

### BRIEF SUMMARY OF THE INVENTION

The present invention relates to novel salts of flunarizine with improved properties. Such improved properties can include, but are not limited to, hygroscopicity, physical stability, chemical stability, and solubility. The invention also provides novel pharmaceutical compositions comprising flunarizine salts, methods of making flunarizine salts, and related methods of treatment. For example, flunarizine benzoate and flunarizine fumarate provide alternatives in a pharmaceutical composition to the currently available form, flunarizine dihydrochloride.

The free form of flunarizine has the following structure (I):

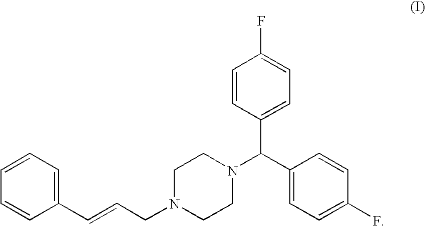

The benzoate and fumarate salts of flunarizine can be used to provide pharmaceutical compositions for the treatment or prevention of conditions known in the art, such as migraine or vertigo.

For a better understanding of the present invention, together with other and further embodiments thereof, reference is made to the accompanying drawings and detailed description.

### BRIEF DESCRIPTION OF THE DRAWINGS

FIG. 1—PXRD diffractogram of flunarizine benzoate

FIG. 2—DSC thermogram of flunarizine benzoate

FIG. 3—TGA thermogram of flunarizine benzoate

FIG. 4—Raman spectrum of flunarizine benzoate

FIG. 5—DVS data of flunarizine benzoate

FIG. 6—PXRD diffractogram of flunarizine fumarate

FIG. 7—DSC thermogram of flunarizine fumarate

FIG. 8—TGA thermogram of flunarizine fumarate

FIG. 9—DVS data of flunarizine fumarate

FIG. 10—DVS comparison of flunarizine dihydrochloride and flunarizine benzoate

### DETAILED DESCRIPTION OF THE INVENTION

The present invention relates to novel salts of flunarizine. The properties of one or more of the novel salts of flunarizine are improved relative to one or more known forms of flunarizine, such as flunarizine free base or flunarizine dihydrochloride (the currently marketed form of flunarizine). Such improved properties can include, but are not limited to, hygroscopicity, physical stability, chemical stability, and solubility. The novel flunarizine salts can take several forms including, but not limited to, hydrates and solvates as well as various stoichiometric ratios of ionized flunarizine to counterion. The invention also includes other forms of flunarizine salts including, but not limited to, polymorphs, co-crystals, and amorphous forms. The invention also provides novel pharmaceutical compositions comprising these forms, methods of making these forms, and related methods of treatment.

The salts of the present invention include flunarizine benzoate and flunarizine fumarate. Flunarizine free base has the following structure (I):

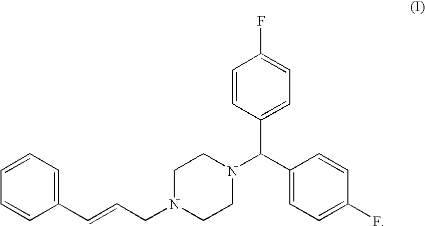

In a first embodiment, the present invention comprises flunarizine benzoate salt. In a second embodiment, the present invention comprises flunarizine fumarate salt.

In a further embodiment, the novel salts of flunarizine can be incorporated into a pharmaceutical composition. In a further embodiment, the novel salts of flunarizine can be incorporated into a controlled release pharmaceutical composition.

In another embodiment, the present salts of flunarizine can be incorporated into a pharmaceutical composition comprising two or more layers of a flunarizine salt of the present invention such that one layer is substantially released prior to the substantial release of another layer in vivo. In another embodiment, the flunarizine salts can be incorporated into a pharmaceutical composition comprising pellets, wherein the pellets have varying extents or compositions of coating so as to enable release of flunarizine salt over a substantially longer period of time than that of the currently available flunarizine dihydrochloride (e.g., SIBELIUM®).

In another embodiment, the present salts of flunarizine can be incorporated into an osmotically active pharmaceutical composition suitable for oral administration. Osmotically active pharmaceutical compositions, osmotic pumps, osmotic drug delivery, and other osmotic technology suitable for oral administration can include, but are not limited to, OROS® Push-Pull and OROS® Tri-layer pharmaceutical compositions. In another embodiment, the present salts of flunarizine can be incorporated into an OROS® drug delivery system. Such controlled release pharmaceutical compositions comprising the present salts of flunarizine, such as an osmotically active pharmaceutical composition suitable for oral administration, may lead to a longer lasting therapeutic effect than that of flunarizine dihydrochloride in the currently available form.

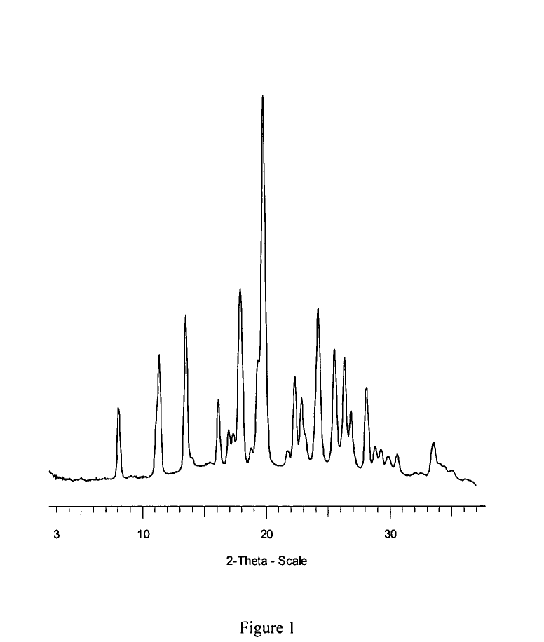

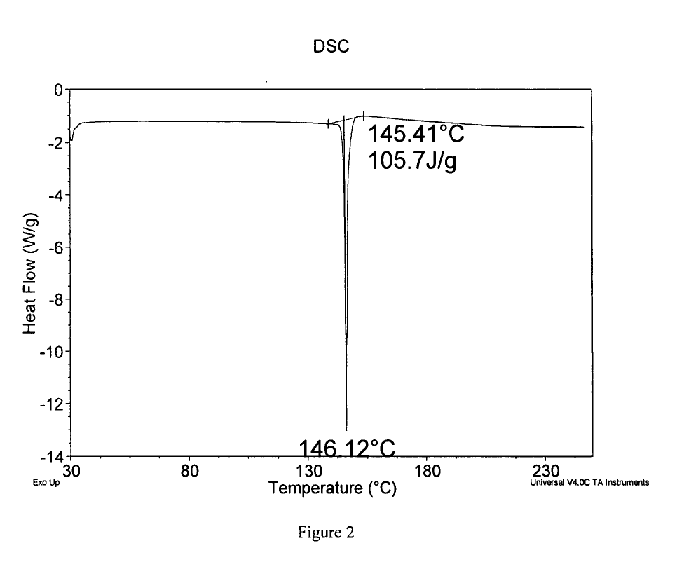

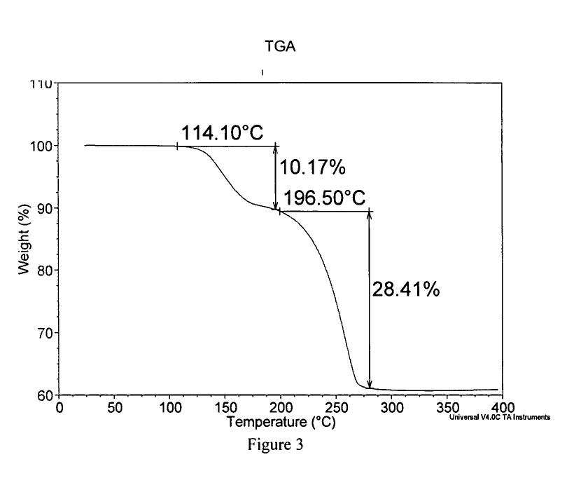

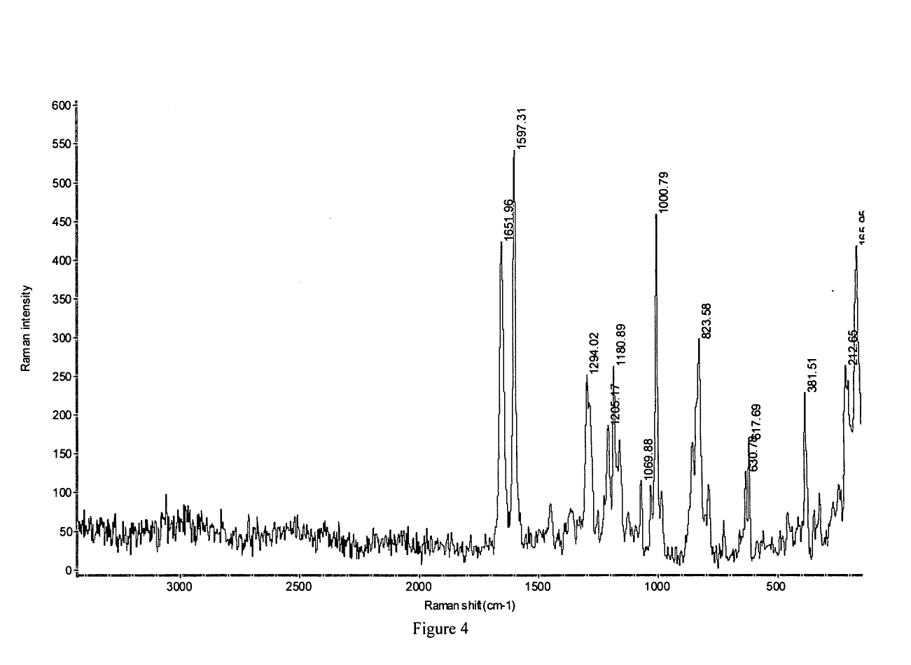

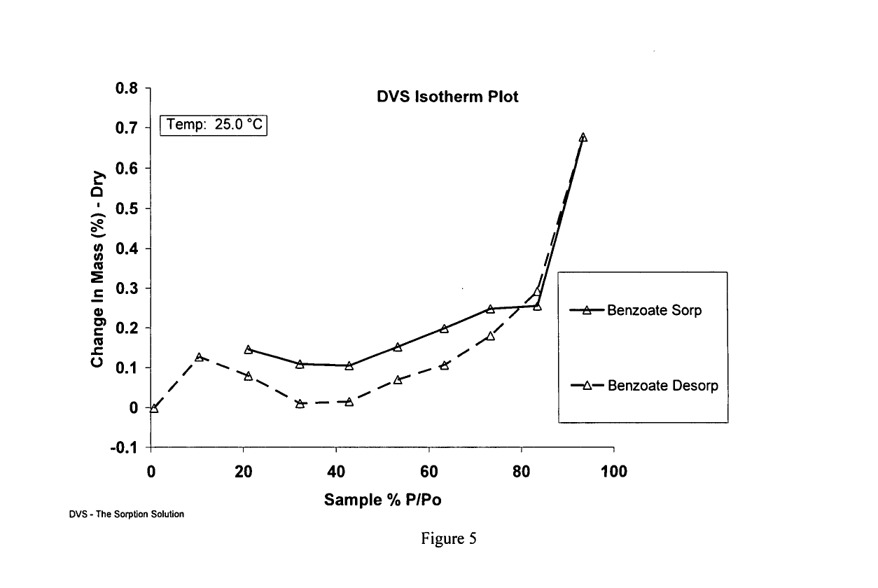

In another embodiment, the present invention comprises flunarizine benzoate. In another embodiment, the present invention comprises flunarizine benzoate, wherein the salt exhibits a PXRD diffractogram comprising a peak at about 8.05 degrees 2-theta. In another embodiment, the present invention comprises flunarizine benzoate, wherein the salt exhibits a PXRD diffractogram comprising a peak at about 11.34 degrees 2-theta. In another embodiment, the present invention comprises flunarizine benzoate, wherein the salt exhibits a PXRD diffractogram comprising a peak at about 13.50 degrees 2-theta. In another embodiment, the present invention comprises flunarizine benzoate, wherein the salt exhibits a PXRD diffractogram comprising peaks at about 8.05 and about 11.34 degrees 2-theta. In another embodiment, the present invention comprises flunarizine benzoate, wherein the salt exhibits a PXRD diffractogram comprising peaks at about 17.92 and about 19.80 degrees 2-theta. In another embodiment, the present invention comprises flunarizine benzoate, wherein the salt exhibits a PXRD diffractogram comprising peaks at about 8.05 and about 13.50 degrees 2-theta. In another embodiment, the present invention comprises flunarizine benzoate, wherein the salt exhibits a PXRD diffractogram comprising peaks at about 8.05, about 11.34, and about 13.50 degrees 2-theta. In another embodiment, the present invention comprises flunarizine benzoate, wherein the salt exhibits a PXRD diffractogram comprising peaks at about 17.92, about 19.80, about 22.32, and about 24.24 degrees 2-theta. In another embodiment, the present invention comprises flunarizine benzoate, wherein the salt exhibits a PXRD diffractogram comprising peaks at about 8.05, about 11.34, about 13.50, about 16.13, and about 17.92 degrees 2-theta. In another embodiment, the present invention comprises flunarizine benzoate, wherein the salt exhibits a PXRD diffractogram comprising peaks at about 16.13, about 17.92, about 19.80, and about 24.24 degrees 2-theta. In another embodiment, the present invention comprises flunarizine benzoate, wherein the salt exhibits a PXRD diffractogram comprising peaks at about 11.34, about 17.92, about 25.57, and about 26.36 degrees 2-theta. In another embodiment, the present invention comprises flunarizine benzoate, wherein the salt exhibits a PXRD diffractogram comprising peaks at about 8.05, about 13.50, about 17.92, about 22.32, and about 25.57 degrees 2-theta. In another embodiment, the present invention comprises flunarizine benzoate, wherein the salt exhibits a PXRD diffractogram comprising peaks at about 8.05, about 11.34, about 13.50, about 16.13, about 17.92, and about 19.80 degrees 2-theta. In another embodiment, the present invention comprises flunarizine benzoate, wherein the salt exhibits a PXRD diffractogram comprising peaks at about 8.05, about 11.34, about 13.50, about 16.13, about 17.92, about 19.80, about 22.32, about 22.89, about 24.24, about 25.57, about 26.36, about 26.87, about 28.15, and about 33.55 degrees 2-theta. In another embodiment, the present invention comprises flunarizine benzoate, wherein the salt exhibits a PXRD diffractogram substantially similar to FIG. 1. In another embodiment, the present invention comprises flunarizine benzoate, wherein the salt exhibits a DSC thermogram comprising an endothermic transition at about 146 degrees C. In another embodiment, the present invention comprises flunarizine benzoate, wherein the salt exhibits a DSC thermogram substantially similar to FIG. 2. In another embodiment, the present invention comprises flunarizine benzoate, wherein the salt exhibits a TGA thermogram substantially similar to FIG. 3. In another embodiment, the present invention comprises flunarizine benzoate, wherein the salt exhibits a Raman spectrum substantially similar to FIG. 4. In another embodiment, the present invention comprises flunarizine benzoate, wherein the salt exhibits dynamic vapor sorption (DVS) characteristics substantially similar to FIG. 5.

In another embodiment, the present invention comprises a flunarizine benzoate salt, wherein the benzoate salt is nonhygroscopic. In another embodiment, the present invention comprises a flunarizine benzoate salt, wherein the benzoate salt is nonhygroscopic from about 10% relative humidity to about 90% relative humidity. In another embodiment, the present invention comprises a flunarizine benzoate salt, wherein the benzoate salt is nonhygroscopic from about 20% relative humidity to about 80% relative humidity. In another embodiment, the present invention comprises a flunarizine benzoate salt, wherein the benzoate salt is nonhygroscopic from about 30% relative humidity to about 70% relative humidity. In another embodiment, the present invention comprises a flunarizine benzoate salt, wherein the benzoate salt is physically stable. In another embodiment, the present invention comprises a flunarizine benzoate salt, wherein the benzoate salt is physically stable from about 10% relative humidity to about 90% relative humidity. In another embodiment, the present invention comprises a flunarizine benzoate salt, wherein the benzoate salt is physically stable from about 20% relative humidity to about 80% relative humidity. In another embodiment, the present invention comprises a flunarizine benzoate salt, wherein the benzoate salt is physically stable from about 30% relative humidity to about 70% relative humidity.

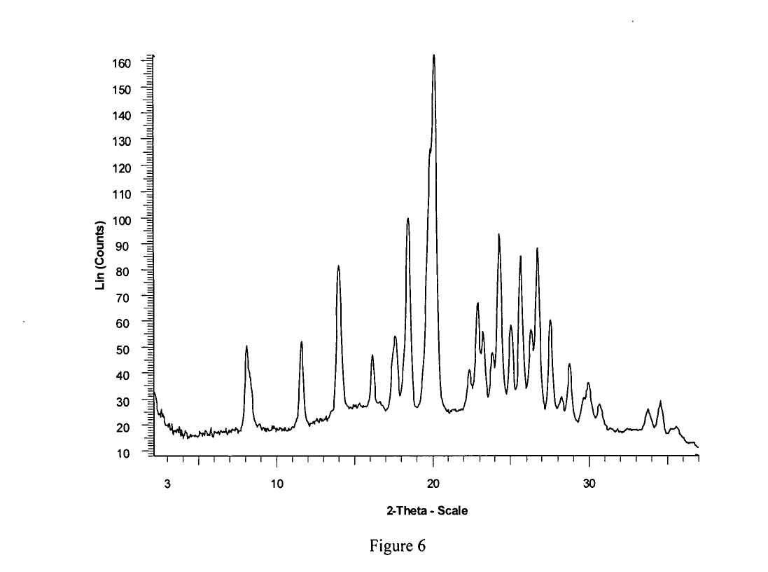

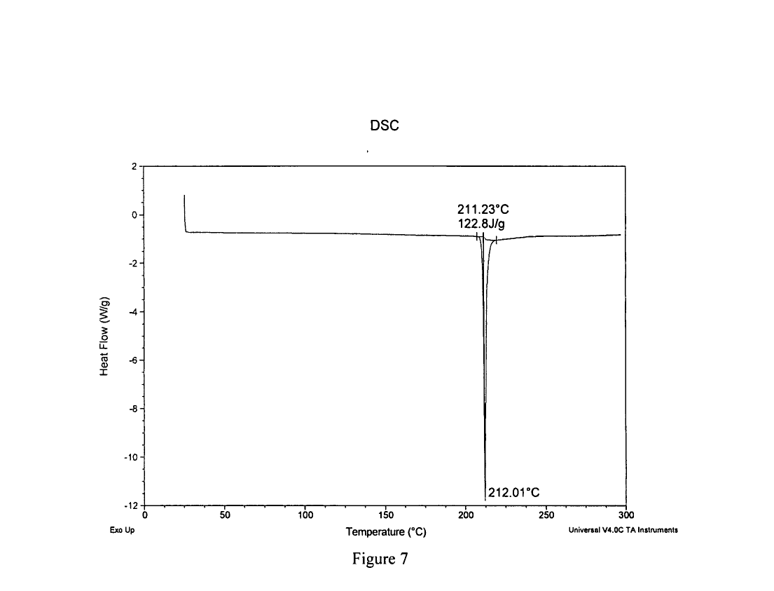

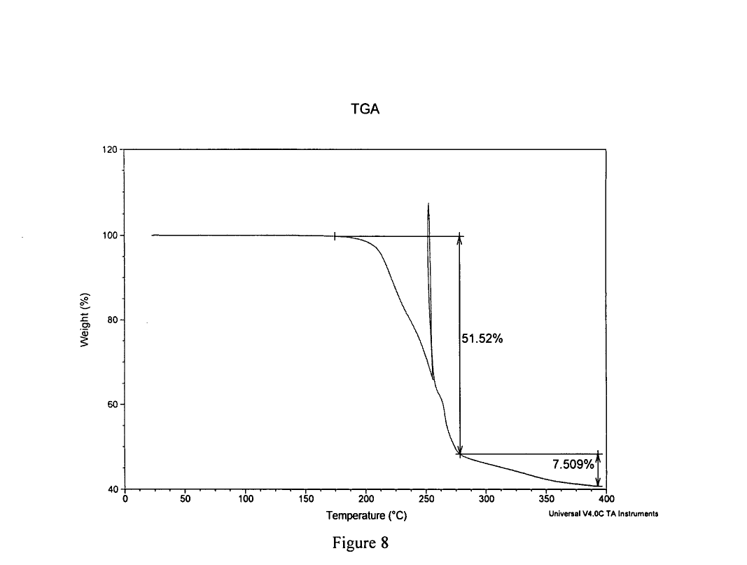

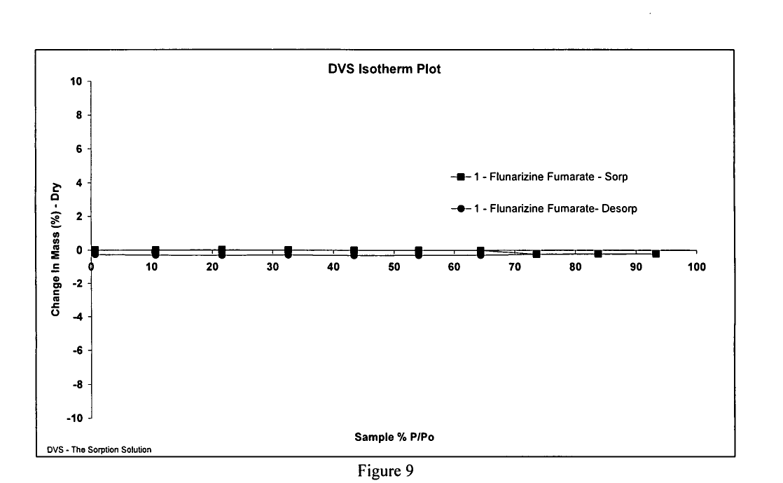

In another embodiment, the present invention comprises flunarizine fumarate. In another embodiment, the present invention comprises flunarizine fumarate, wherein the salt exhibits a PXRD diffractogram comprising a peak at about 8.06 degrees 2-theta. In another embodiment, the present invention comprises flunarizine fumarate, wherein the salt exhibits a PXRD diffractogram comprising a peak at about 11.56 degrees 2-theta. In another embodiment, the present invention comprises flunarizine fumarate, wherein the salt exhibits a PXRD diffractogram comprising a peak at about 13.98 degrees 2-theta. In another embodiment, the present invention comprises flunarizine fumarate, wherein the salt exhibits a PXRD diffractogram comprising peaks at about 8.06 and about 13.98 degrees 2-theta. In another embodiment, the present invention comprises flunarizine fumarate, wherein the salt exhibits a PXRD diffractogram comprising peaks at about 8.06 and about 11.56 degrees 2-theta. In another embodiment, the present invention comprises flunarizine fumarate, wherein the salt exhibits a PXRD diffractogram comprising peaks at about 11.56 and about 13.98 degrees 2-theta. In another embodiment, the present invention comprises flunarizine fumarate, wherein the salt exhibits a PXRD diffractogram comprising peaks at about 8.06, about 11.56, and about 13.98 degrees 2-theta. In another embodiment, the present invention comprises flunarizine fumarate, wherein the salt exhibits a PXRD diffractogram comprising peaks at about 8.06, about 13.98, about 18.46, and about 20.16 degrees 2-theta. In another embodiment, the present invention comprises flunarizine fumarate, wherein the salt exhibits a PXRD diffractogram comprising peaks at about 11.56, about 16.14, about 17.63, about 18.46, and about 20.16 degrees 2-theta. In another embodiment, the present invention comprises flunarizine fumarate, wherein the salt exhibits a PXRD diffractogram comprising peaks at about 8.06, about 11.56, about 13.98, and about 16.14 degrees 2-theta. In another embodiment, the present invention comprises flunarizine fumarate, wherein the salt exhibits a PXRD diffractogram comprising peaks at about 8.06, about 13.98, about 24.31, and about 25.67 degrees 2-theta. In another embodiment, the present invention comprises flunarizine fumarate, wherein the salt exhibits a PXRD diffractogram comprising peaks at about 11.56, about 17.63, about 19.86, about 22.90, and about 24.31 degrees 2-theta. In another embodiment, the present invention comprises flunarizine fumarate, wherein the salt exhibits a PXRD diffractogram comprising peaks at about 8.06, about 11.56, about 13.98, about 16.14, about 17.63, and about 18.46 degrees 2-theta. In another embodiment, the present invention comprises flunarizine fumarate, wherein the salt exhibits a PXRD diffractogram comprising peaks at about 8.06, about 11.56, about 13.98, about 16.14, about 17.63, about 18.46, about 19.86, about 20.16, about 22.90, about 24.31, about 25.67, about 26.76, about 27.60, and about 28.80 degrees 2-theta. In another embodiment, the present invention comprises flunarizine fumarate, wherein the salt exhibits a PXRD diffractogram substantially similar to FIG. 6. In another embodiment, the present invention comprises flunarizine fumarate, wherein the salt exhibits a DSC thermogram comprising an endothermic transition at about 212 degrees C. In another embodiment, the present invention comprises flunarizine fumarate, wherein the salt exhibits a DSC thermogram substantially similar to FIG. 7. In another embodiment, the present invention comprises flunarizine fumarate, wherein the salt exhibits a TGA thermogram substantially similar to FIG. 8. In another embodiment, the present invention comprises flunarizine fumarate, wherein the salt exhibits dynamic vapor sorption (DVS) characteristics substantially similar to FIG. 9.

In another embodiment, the present invention comprises a flunarizine fumarate salt, wherein the fumarate salt is nonhygroscopic. In another embodiment, the present invention comprises a flunarizine fumarate salt, wherein the fumarate salt is nonhygroscopic from about 10% relative humidity to about 90% relative humidity. In another embodiment, the present invention comprises a flunarizine fumarate salt, wherein the fumarate salt is nonhygroscopic from about 20% relative humidity to about 80% relative humidity. In another embodiment, the present invention comprises a flunarizine fumarate salt, wherein the fumarate salt is nonhygroscopic from about 30% relative humidity to about 70% relative humidity. In another embodiment, the present invention comprises a flunarizine fumarate salt, wherein the fumarate salt is physically stable. In another embodiment, the present invention comprises a flunarizine fumarate salt, wherein the fumarate salt is physically stable from about 10% relative humidity to about 90% relative humidity. In another embodiment, the present invention comprises a flunarizine fumarate salt, wherein the fumarate salt is physically stable from about 20% relative humidity to about 80% relative humidity. In another embodiment, the present invention comprises a flunarizine fumarate salt, wherein the fumarate salt is physically stable from about 30% relative humidity to about 70% relative humidity.

According to the present invention, the present flunarizine salts can have various stoichiometric ratios of ionized flunarizine (cation) to counterion (anion). For example, the ratio of cation:anion can be 1:1 or 2:1. Other stoichiometric ratios are also included in the invention.

In another embodiment, the present invention comprises flunarizine salts, and methods of making and using the same. In another embodiment, the present invention comprises a hydrate of one or more flunarizine salts. In another embodiment, the present invention comprises a solvate of one or more flunarizine salts. In another embodiment, the present invention comprises one or more polymorphs of a salt of flunarizine or one or more polymorphs of a hydrate or a solvate of a salt of flunarizine. In another embodiment, the present invention comprises a co-crystal of one or more flunarizine salts. In another embodiment, the present invention comprises an amorphous form of one or more flunarizine salts, and methods of making and using the same.

In another embodiment, a flunarizine salt form can exist in a form such as, but not limited to, an anhydrous form, a hydrate form, a dehydrate form, or a solvate form. Such hydrate and solvate forms can have various stoichiometric ratios of ionized flunarizine to water or solvate molecules such as, but not limited to, 1:1, 1:1.5, 2:1, or 1:2.

In another embodiment, the present invention provides a method of making an organic acid salt of flunarizine, comprising:
(a) providing flunarizine free base; and
(b) contacting said flunarizine free base with an appropriate organic acid so as to crystallize said salt of flunarizine.

In a specific embodiment, said appropriate organic acid is benzoic acid. In another specific embodiment, said appropriate organic acid is fumaric acid. In another embodiment, a solvent is added to said flunarizine free acid prior to said contact with an appropriate organic acid. In another embodiment, step (b) is completed in the presence of a solvent such that a solution is formed prior to crystallization of the salt of flunarizine. In another embodiment, step (b) is completed in the presence of a solvent such that a suspension is formed prior to crystallization of the salt of flunarizine. In certain embodiments, a solvent is selected from the group consisting of: acetone, ethanol, nitromethane, methanol, acetonitrile, dichloromethane, water, 2-propanol, isopropyl acetate, and tetrahydrofuran (THF). In another embodiment, a solvent comprises a mixture of any two or more solvents, including, but not limited to, acetone, ethanol, nitromethane, methanol, acetonitrile, dichloromethane, water, 2-propanol, isopropyl acetate, and tetrahydrofuran.

Flunarizine free base and flunarizine dihydrochloride can be prepared by one or more methods available in the art, including, but not limited to, the method in U.S. Pat. No. 3,773,939.

In one embodiment of the present invention, an amount of a flunarizine salt of the invention effective to modulate a mammal's physiology and/or to treat a mammal is administered to said mammal. In one aspect, the flunarizine salt is administered in an amount sufficient to effect modulation of a mammal's physiology and/or treatment.

In another embodiment, a method of treating a mammal suffering from vertigo is provided, comprising administering to said mammal an effective amount of a flunarizine salt. In another embodiment, a method of preventing a mammal from suffering from migraine is provided, comprising administering to said mammal an effective amount of a flunarizine salt. In another embodiment, a method of preventing a mammal from suffering from classic (with aura) migraine is provided, comprising administering to said mammal an effective amount of a flunarizine salt. In another embodiment, a method of preventing a mammal from suffering from common (without aura) migraine is provided, comprising administering to said mammal an effective amount of a flunarizine salt. In another embodiment, a method of treating a mammal suffering from peripheral vascular disease, refractory epilepsy resistant to conventional antiepileptic therapy, or alternating hemiplegia of childhood is provided, comprising administering to said mammal an effective amount of a flunarizine salt. In another embodiment, said mammal is a human.

In another embodiment, the present invention includes the preparation of a medicament comprising a salt of flunarizine. Such a medicament can be used for treating or preventing vertigo, vestibular vertigo, migraine, classic migraine, common migraine, peripheral vascular disease, refractory epilepsy resistant to conventional antiepileptic therapy, or alternating hemiplegia of childhood in a mammal in need of such treatment or prevention. In another embodiment, said mammal is a human.

Pharmaceutical dosage forms of flunarizine salt can be administered in several ways including, but not limited to, oral administration. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also be provided by the present invention, including such non-limiting examples as a suspension, solution, syrup, or emulsion.

Flunarizine salts can be administered by controlled or delayed release means.

Typical daily dosage forms of the invention comprise flunarizine salt, in an amount of from about 1.0 mg to about 25.0 mg, from about 2.5 mg to 20.0 mg, or from about 5.0 mg to about 15.0 mg. In a particular embodiment, the flunarizine salt for use in such a composition is flunarizine benzoate or flunarizine fumarate. The dosage amounts described herein are expressed in amounts of flunarizine free base and do not include the weight of a counterion (e.g., benzoate or fumarate) or any water or solvent molecules.

In another embodiment of the invention, a pharmaceutical composition comprising flunarizine salt is administered orally as needed in an amount of from about 5.0 mg to about 20.0 mg, from about 5.0 mg to about 15.0 mg, or from about 5.0 mg to about 10.0 mg flunarizine. For example, about 5.0 mg, about 7.5 mg, or about 10.0 mg. In specific embodiments, pharmaceutical compositions comprising flunarizine salt can be administered orally in amounts of about 5.0 mg or about 10.0 mg. The dosage amounts can be administered in single or divided doses. In another embodiment, a daily dose of a pharmaceutical composition comprising flunarizine salt comprises up to about 20.0 mg flunarizine. In other embodiments, the present invention is directed to compositions comprising flunarizine salt as described herein and one or more diluents, carriers, and/or excipients suitable for the administration to a mammal for the treatment or prevention of one or more of the conditions described herein.

The flunarizine salts of the present invention may also be used to prepare pharmaceutical dosage forms other than the oral dosage forms described above, such as topical dosage forms, parenteral dosage forms, transdermal dosage forms, and mucosal dosage forms. For example, such forms include creams, lotions, solutions, suspensions, emulsions, ointments, powders, patches, suppositories, and the like.

The flunarizine salt forms of the present invention can be characterized, e.g., by the TGA, DSC, DVS, Raman spectroscopy, single crystal x-ray diffractometer data, or by any one, any two, any three, any four, any five, any six, any seven, any eight, any nine, any ten, or any single integer number of PXRD 2-theta angle peaks, or by any combination of the data acquired from the analytical techniques described herein.

Although the invention has been described with respect to various embodiments, it should be realized this invention is also capable of a wide variety of further and other embodiments within the spirit and scope of the appended claims.

### EXAMPLES

### Example 1

### Flunarizine benzoate (1-cinnamyl-4-(di-p-fluorobenzhydryl)piperazine benzoate)

In a vial with stirring, 807.1 mg of flunarizine free base (2.00 mmol) was completely dissolved with 4 mL of acetone and passed through a PTFE filter. To this solution, a solution of 274.3 mg of benzoic acid (2.44 mmol) in 1 mL of acetone was added in aliquots (4×0.25 mL). After a few minutes a crystalline solid started to precipitate and the suspension was left overnight with stirring at room temperature. Upon stirring overnight at room temperature, the suspension was filtered under vacuum through a filter paper on a Buchner funnel and the solid was left to air dry in an open container.

Crystals representative of those obtained by completing the method above were analyzed using PXRD, DSC, TGA, Raman, dynamic vapor sorption (DVS), and single crystal x-ray diffraction. The flunarizine benzoate exhibits a PXRD diffractogram comprising peaks, for example, at about 8.05, 11.34, 13.50, 16.13, 16.97, 17.30, 17.92, 18.79, 19.34, 19.80, 21.74, 22.32, 22.89, 23.22, 24.24, 25.57, 26.36, 26.87, 28.15, 28.83, 29.30, 29.88, 30.61 and about 33.55 degrees 2-theta (See FIG. 1). DSC showed an endothermic transition at about 146 degrees C. (See FIG. 2). TGA showed the flunarizine benzoate lost about 10 percent weight between about 115 degrees C. and about 190 degrees C. and lost about 28 percent weight between about 197 degrees C. and about 295 degrees C. (See FIG. 3). The flunarizine benzoate exhibits a Raman spectrum comprising peaks, for example, at about 1652, 1597, 1294, 1205, 1181, 1070, 1001, 824, 631, 618, 382, 213, and about 166 cm⁻¹ (See FIG. 4).

Dynamic vapor sorption (DVS) analysis was completed on flunarizine benzoate from about 0 to about 95 percent relative humidity at about 25 degrees C. (See FIG. 5). The DVS analysis showed flunarizine benzoate is stable from about 0% to about 95% relative humidity. This is characteristic of a nonhygroscopic material.

Single crystal data: C₃₃H₃₂F₂N₂O₂, M=526.61, monoclinic, Pc, a=10.7748(13) angstroms, b=10.9190(13) angstroms, c=11.3612(14) angstroms, alpha=90 degrees, beta=93.221(6) degrees, gamma=90 degrees, T=100(2) K, Z=3, D_{c}=1.310 Mg/m³, V=1334.5(3) cubic angstroms, λ=0.71073 angstroms.

### Example 2

### Flunarizine fumarate (1-cinnamyl-4-(di-p-fluorobenzhydryl)piperazine fumarate)

In a vial with stirring, 2.5020 g of flunarizine free base (6.18 mmol) were completely dissolved with 16 mL of acetone and passed through a PTFE filter. To this solution, a solution of 0.911 g of fumaric acid (7.85 mmol) in 12 mL of ethanol plus 0.5 mL of water, was added dropwise (0.25 mL/min) with stirring. After addition of about 1 mL of the fumaric acid solution, the formation of precipitate was observed. After finishing the addition, the suspension was left overnight with stirring at room temperature. The next day, the suspension was filtered under vacuum through a filter paper supported on a stainless steel grid and washed with 10 mL of cold 50:50 acetone:ethanol. The solid was left to air dry on the filter and later on an open tray. Afterwards, the solid was transferred to a vial and stored at room temperature.

Crystals representative of those obtained by completing the method above were analyzed using PXRD, DSC, TGA, dynamic vapor sorption (DVS), and single crystal x-ray diffraction. The flunarizine fumarate exhibits a PXRD diffractogram comprising peaks, for example, at about 8.06, 11.56, 13.98, 16.14, 17.63, 18.46, 19.86, 20.16, 22.39, 22.90, 23.29, 23.86, 24.31, 25.07, 25.67, 26.36, 26.76, 27.60, 28.29, 28.80, 30.02, 30.73, 33.85 and about 34.61 degrees 2-theta (See FIG. 6). DSC showed an endothermic transition at about 212 degrees C. (See FIG. 7). TGA showed the flunarizine fumarate lost about 52 percent weight between about 175 degrees C. and about 275 degrees C. and lost about 8 percent weight between about 275 degrees C. and about 400 degrees C. (See FIG. 8).

Dynamic vapor sorption (DVS) analysis was completed on flunarizine fumarate from about 0 to about 95 percent relative humidity at about 25 degrees C. (See FIG. 9). The DVS analysis showed flunarizine fumarate is stable from about 0% to about 95% relative humidity. This is characteristic of a nonhygroscopic material.

Single crystal data: C₃₀H₃₀F₂N₂O₄, M=520.56, monoclinic, Pc, a=10.8336(9) angstroms, b=10.5327(10) angstroms, c=11.2238(10) angstroms, alpha=90 degrees, beta=91.087(5) degrees, gamma=90 degrees, T=140(2) K, Z=2, D_{c}=1.350 Mg/m³, V=1280.5(2) cubic angstroms, λ=0.71073 angstroms.

The flunarizine salts prepared according to Examples 1 and 2 were analyzed using the following analytical techniques:

### Differential Scanning Calorimetry

DSC analysis of each sample was performed using a Q1000 Differential Scanning Calorimeter (TA Instruments, New Castle, Del., U.S.A.), which uses Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©2001 TA Instruments-Water LLC), or another version as specified in the drawings or otherwise herein.

For all of the DSC analyses, an aliquot of a sample was weighed into either a standard aluminum pan (Pan part #900786.091; lid part #900779.901) or a hermetic aluminum pan (Pan part #900793.901; lid part #900794.901 (TA Instruments, New Castle Del. USA)). Non-solvated samples were loaded into standard pans and were sealed either by crimping for dry samples or press fitting for wet samples (such as slurries). Solvated samples (including hydrates) were loaded into hermetic pans and hermetically sealed. The sample pan was loaded into the Q1000 Differential Scanning Calorimeter, which is equipped with an autosampler, and a thermogram was obtained by individually heating the same using the control software at a rate of 10° C./minute from Tₘᵢₙ (typically 30° C.) to Tₘₐₓ (typically 300° C.) using an empty aluminum pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

### Thermogravimetric Analysis

Thermogravimetric analysis (TGA) of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.1E; Build 3.1.0.40 (2001 TA Instruments-Water LLC), or another version as specified in the drawings or otherwise herein.

For the TGA experiments, the purge gas used was dry nitrogen, the balance purge was 40 mL/minute N₂, and the sample purge was 60 mL/minute N₂.

TGA was performed on the sample by placing a sample of the flunarizine salt in a platinum pan. The starting temperature was typically 20 degrees C. with a heating rate of 10 degrees C./minute, and the ending temperature was 300 degrees C.

### Powder X-ray Diffraction

Powder x-ray diffraction patterns were obtained using a Bruker D8 Discover with GADDS diffractometer (Bruker-AXS Inc., Madison, Wis., U.S.A).

The Bruker D8 Discover with GADDS Diffractometer was equipped with a copper source (Cu/K_{α}1.5406Å), computer controlled x-y-z stage, a 0.5 mm collimator and a Hi-Star area detector. Samples were loaded into a proprietary sample holder by tapping the sample holder into a powder bed and arraying the holders into a 96 position block. The block was then loaded onto the x-y-z stage and the sample positions were entered into the software. A diffractogram was acquired using control software (GADDS—General Area Detector Diffraction System, (Bruker, version 4.1.14 (©1997-2003 Bruker-AXS.)) under ambient conditions at a power setting of 46 kV at 40 mA in reflectance mode. The exposure time was 5 minutes unless otherwise specified. The diffractogram obtained was integrated of 2-theta from 2-40 degrees and chi (1 segment) from 0-36 degrees at a step size of 0.02 degrees using the GADDS software.

The relative intensity of peaks in a diffractogram is not necessarily a limitation of the PXRD pattern because peak intensity can vary from sample to sample, e.g., due to crystalline impurities. Further, the angles of each peak can vary by about ±0.1 degrees, or by about ±0.05. The entire pattern or most of the pattern peaks may also shift by about ±0.1 degrees to about ±0.2 degrees due to differences in calibration, settings, and other variations from instrument to instrument and from operator to operator. All reported PXRD peaks in the Figures, Examples, and elsewhere herein are reported with an error of about ±0.1 degrees 2-theta. Unless otherwise noted, all diffractograms are obtained at about room temperature (about 24 degrees C. to about 25 degrees C.).

### Single Crystal X-ray Analysis

Single crystal x-ray data were collected on a Bruker Kappa-APEX II CCD diffractometer. Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemans Analytical X-ray Instruments Inc.: Madison, Wis.).

### Example 3

### Comparative Dynamic Vapor Sorption (DVS) Data for Flunarizine Dihydrochloride

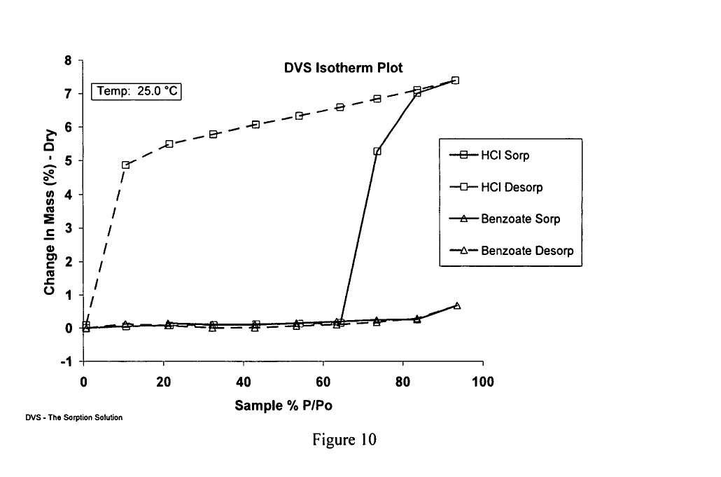

FIG. 10 shows comparative DVS data of flunarizine dihydrochloride (at about 25 degrees C.) overlayed with the DVS data for flunarizine benzoate. Flunarizine dihydrochloride is hygroscopic. The dihydrochloride salt rapidly adsorbs water (greater than 7 percent change in mass) at relative humidity levels above about 60 percent. The release of the adsorbed water is difficult upon desorption. In comparison, the flunarizine benzoate salt of the present invention adsorbs relatively little water (less than 1 percent change in mass) at relative humidity levels up to about 95 percent.

### Example 4

### Excipient Compatability Data for Flunarizine Dihydrochloride and Flunarizine Benzoate

Two solid mixes, one containing sucrose:crospovidone:flunarizine dihydrochloride 94:5:1 (weight %), and the other containing sucrose:crospovidone:flunarizine benzoate 94:5:1 (weight %), were prepared in mortars, transferred to separate vials and a small amount of water was added until a very viscous syrup was formed. The samples were left to air dry at room temperature for two days followed by one day under vacuum at 40 degrees C. After the processing was finished, pictures of the samples were taken. Vials with identical content to the ones pictured were seal crimped and stored at 60 degrees C. for 2 weeks. After this period, their pictures were taken. Comparison of both sets of images shows substantially more degradation had occurred in the mixes containing flunarizine dihydrochloride than those containing flunarizine benzoate.

What is claimed is:

1. 1-cinnamyl-4-(di-p-fluorobenzhydryl)piperazine benzoate salt.

2. The 1-cinnamyl-4-(di-p-fluorobenzhydryl)piperazine benzoate salt of claim 1, wherein said benzoate salt exhibits a powder X-ray diffractogram comprising a peak at about 8.05 degrees 2-theta.

3. The 1-cinnamyl-4-(di-p-fluorobenzhydryl)piperazine benzoate salt of claim 1, wherein said benzoate salt exhibits a powder X-ray diffractogram comprising a peak at about 11.34 degrees 2-theta.

4. The 1-cinnamyl-4-(di-p-fluorobenzhydryl)piperazine benzoate salt of claim 1, wherein said benzoate salt exhibits a powder X-ray diffractogram comprising peaks at about 8.05 and about 13.50 degrees 2-theta.

5. The 1-cinnamyl-4-(di-p-fluorobenzhydryl)piperazine benzoate salt of claim 1, wherein said benzoate salt exhibits a powder X-ray diffractogram comprising peaks at about 11.34, about 13.50, and about 16.13 degrees 2-theta.

6. The 1-cinnamyl-4-(di-p-fluorobenzhydryl)piperazine benzoate salt of claim 1, wherein said benzoate salt exhibits a powder X-ray diffractogram comprising peaks at about 8.05, about 11.34, about 13.50, about 16.13, about 17.92, about 19.80, about 22.32, about 22.89, about 24.24, about 25.57, about 26.36, and about 28.15 degrees 2-theta.

7. The 1-cinnamyl-4-(di-p-fluorobenzhydryl)piperazine benzoate salt of claim 1, wherein said benzoate salt exhibits a DSC thermogram comprising an endothermic transition at about 146 degrees C.

8. The 1-cinnamyl-4-(di-p-fluorobenzhydryl)piperazine benzoate salt of claim 1, wherein said benzoate salt is nonhygroscopic from about 10% relative humidity to about 90% relative humidity.

9. A method of making an organic acid salt of 1-cinnamyl-4-(di-p-fluorobenzhydryl)piperazine, comprising:
(a) providing 1-cinnamyl-4-(di-p-fluorobenzhydryl)piperazine; and
(b) contacting said 1-cinnamyl-4-(di-p-fluorobenzhydryl)piperazine with an appropriate organic acid so as to crystallize said salt of 1-cinnamyl-4-(di-p-fluorobenzhydryl)piperazine.

10. The method of claim 9, wherein said appropriate organic acid is benzoic acid.

11. A method of treating a mammal suffering from vertigo, comprising administering to said mammal an effective amount of the 1-cinnamyl-4-(di-p-fluorobenzhydryl)piperazine benzoate salt of claim 1.

12. A method of preventing a mammal from suffering from migraine, common migraine, or classic migraine, comprising administering to said mammal an effective amount of the 1-cinnamyl-4-(di-p-fluorobenzhydryl)piperazine benzoate salt of claim 1.

13. 1-cinnamyl-4-(di-p-fluorobenzhydryl)piperazine fumarate salt.

14. The 1-cinnamyl-4-(di-p-fluorobenzhydryl)piperazine fumarate salt of claim 13, wherein said fumarate salt exhibits a powder X-ray diffractogram comprising a peak at about 8.06 degrees 2-theta.

15. The 1-cinnamyl-4-(di-p-fluorobenzhydryl)piperazine fumarate salt of claim 13, wherein said fumarate salt exhibits a powder X-ray diffractogram comprising a peak at about 13.98 degrees 2-theta.

16. The 1-cinnamyl-4-(di-p-fluorobenzhydryl)piperazine fumarate salt of claim 13, wherein said fumarate salt exhibits a powder X-ray diffractogram comprising peaks at about 8.06 and about 13.98 degrees 2-theta.

17. The 1-cinnamyl-4-(di-p-fluorobenzhydryl)piperazine fumarate salt of claim 13, wherein said fumarate salt exhibits a powder X-ray diffractogram comprising peaks at about 16.14, about 17.63, and about 18.46 degrees 2-theta.

18. The 1-cinnamyl-4-(di-p-fluorobenzhydryl)piperazine fumarate salt of claim 13, wherein said fumarate salt exhibits a powder X-ray diffractogram comprising peaks at about 8.06, about 11.56, about 13.98, about 16.14, about 17.63, about 18.46, about 20.16, about 22.90, about 24.31, about 25.67, about 26.76, and about 27.60 degrees 2-theta.

19. The 1-cinnamyl-4-(di-p-fluorobenzhydryl)piperazine fumarate salt of claim 13, wherein said fumarate salt exhibits a DSC thermogram comprising an endothermic transition at about 212 degrees C.

20. The 1-cinnamyl-4-(di-p-fluorobenzhydryl)piperazine fumarate salt of claim 13, wherein said fumarate salt is nonhygroscopic from about 10% relative humidity to about 90% relative humidity.

21. The method of claim 9, wherein said appropriate organic acid is fumaric acid.

22. A method of treating a mammal suffering from vertigo, comprising administering to said mammal an effective amount of the 1-cinnamyl-4-(di-p-fluorobenzhydryl)piperazine fumarate salt of claim 13.

23. A method of preventing a mammal from suffering from migraine, common migraine, or classic migraine, comprising administering to said mammal an effective amount of the 1-cinnamyl-4-(di-p-fluorobenzhydryl)piperazine fumarate salt of claim 13.
